(19) United States
(12) Patent Application Publication
(10) Pub. No.: US 2002/0139373 A1
(43) Pub. Date: Oct. 3, 2002

(54) Secure condom

(76) Inventor: Cleon Louis Griffiths, Lakewood, CO (US)

Correspondence Address: Cleon L. Griffiths, 10080 West 8Th Place, Lakewood, CO, 80215, US

(21) Appl. No.: 09/825,483

(22) Filed: Apr. 3, 2001

(51) Int. Cl. A61F 6/04

(52) U.S. Cl. 128/844

(57) ABSTRACT

Visualize a condom shaped like a lollipop, the part resembling the stick, of smaller circumference than the shaft of the male member it fits, said smaller circumference will not fit easily over the head of said male member, but will require a small amount of effort to remove said condom which will not accidentally come off, even when lubricated, an installation ring is neccesary to install said condom with said small circumferance area rolled up and stretched onto a groove on the outside circumference of said ring, said condoms and said rings will come in various sizes, the part of said condom resembling the candy part of said lollipop will fit loosely around the head of said male member, preventing splitting and enhansing the sensation of sexual intercourse since enlarged area does not bind the sensitive Glans Penis of male member, free cardboard gauges are available determining correct size for user.

### BACKGROUND-FIELD OF INVENTION

This invention relates to Condoms, specifically to an improved Condom that is designed to remain securely on male member until forcibly removed.

### BACKGROUND-DESCRIPTION OF PRIOR ART

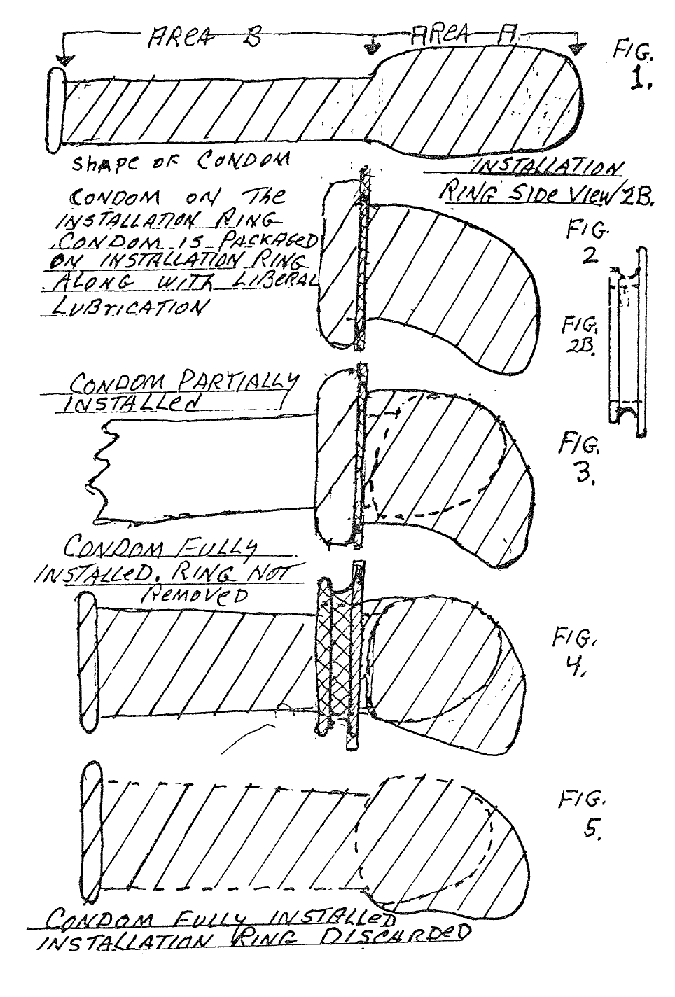

Condoms are made of very thin rubber or plastic in the form of tube that is closed on one end and open on the other. The tube is uniform in diameter for the entire length. The purpose of a Condom is to prevent unwanted pregnancies and prevent the spread of venereal diseases. The Condoms of prior art have several drawbacks. One drawback is the fact that if Condoms are used without lubrication inside, the sensation of intercourse is like going wading in a puddle with overshoes on. The advantage of using a condom dry is that it will likely remain on the penis thruout sexual intercourse. If a condom has lubrication inside The sensation will be much better, but the condom is likely to come off during intercourse which would cancel out all of the benefits that you were using the condom for in the first place. The Secure Condom addresses both the problem of sensation with lubrication and an enlarged closed end for comfort, then it has the remainder of the condom too small to easily fit over the head of the particular penis it fits.(these come in various sizes and are packaged in lubricant.) Secure Condoms come with the small diameter area rolled and stretched upon an Install-ation ring. (FIG. 2) This ring is made of plastic or some other durable material. The outside circumferance of the inst-allation ring has a groove to accept the rolled condom. One side of the installation ring is of larger circumferance to prevent the condom from being rolled off the wrong side of the ring. The inside of the installation ring is smooth and of large enough diameter to allow a penis to enter thru the ring into the enlarged area. (FIG. 3) To use this condom, it is removed from the package. The Secure condom is attached to the installation ring prior to packaging. Identifying the side of the ring that has the largest circumferance tells the user that this is the front of the condom. The penis is pushed thru the installation ring into the enlarged area of the condom at this tine the area of the condom rolled up on the ring is unrolled to the base of the penis. (FIG. 4) The installation ring is then slid over the head of the penis and discarded. (FIG. 5) The area B of the condom is smaller in diameter than the penis that it fits, and will not go over the head of the penis without some effort. Sexual intercourse will not cause area B of the condom to go over the head of the penis. The fit on the shaft of the penis will be tight, but not uncomfortable. It is for this reason that the name of these condoms is SECURE CONDOMS BECAUSE THEY WILL NOT COME OFF DURING INTERCOURSE.. The second advantage to the Secure Condom is that the sensitive area of the Penis known as the Glans Penis is in an area of the condom that is not the least bit restrictive and is bathed in lubricant similar to sexual intercourse without a condom.

The Glans Penis can slide around in the enlarged area of the Secure Condom with the sensitivity of having sexual intercourse without a condom, and without the worry of the condom coming off or splitting as some too tight condoms are prone to do. The following prior art patents have been found to be revelant to the field of the present invention.

U.S. Pat. No. 4,821,742 Issued to Phelps on Apr. 18, 1989 for “Con-traceptive Device” Hereafter the “Phelps Patent”

U.S. Pat. No. 4,966,166 issued to Leffler on Oct. 30 1990 for Proph-ylactic device and Method” Hereafter the “Leffler Patent”

U.S. Pat. No. 5,010,871 Issued to Christina on Apr. 30, 1991 for “Prophylactic Safety Device” Hereafter the “Christina Patent”

U.S. Pat. No. 5,111,831 issued to Foggia on May 12, 1992 for “Scrotum Supporting Condom with Retention Means” Hereafter the “Foggia Patent”.

U.S. Pat. No. 5,199,444 issued to Wheeler on Apr. 6, 1993 for “Condom having enhanced Grippability structure and annular sealing element” Hereafter the “Wheeler Patent”.

U.S. Pat. No. 5,284,159 issued to Wilk on Feb. 8, 1994 for “Pro-phylactic Device” Hereafter the “Wilk Patent”.

The Phelps Patent is a cap that is attached to the head of a penis with adhesive. The main drawback is the pain of removing this cap from a very sensitive area. Also the penis is very seldom completely dry and would not hold adhesive very well so the possibility of the cap coming off during intercourse is great. This is one of the better features of the Secure Condom is that it will stay or a penis during intercourse without the use of adhesive or straps or other contrivances.

The Leffler Invention has the advantage of having more sensi-tivity than regular condors and would probably stay on, but it is complicated to make and use. There is a statement in the claims that shows a definite weakness in the use of this condom. “The device of claim 1 wherein the inside of the mid-section of the device is lubricated and wherein the inside edge of the sleeve section is closed and releasably sealed to itself thereby separating the lubricated inside of the midsection from the inside surface of the sleeve section.” This problem of a condom being lubricated in part but not everywhere would make it impossible to lubricate it before packaging without the lubricate getting all over the condom. There could also be a problem with installing this condom thru these sleeves. With the Secure Con-dom the manufacture of it would be no more difficult than a regular condom of prior art. The Secure Condom can be packaged in water soluable lubricant so that it is lubricated all over and not just in a certain area, which makes it much simpler to make. With the smaller circumferance of the Secure Condom that fits the shaft of the penis tightly the user will have the advantage of having the sensitivity of lubrication inside and outside with no worry of the condom coming off or leaking.

U.S. Pat. No. 5,010,871 issued to Christina on Apr. 30, 1991 for “Prophylactic Safety Device” This is a ring that is apparently slid on the condom and onto the base of the penis. It is pack-aged with this ring attached to a corner of the open end of the condom by a strip of similar material as the condom. This Con-dom does not address the movement of the head of the penis in an enlarged lubricated area such as the Secure Condom does, but only keeps the condom from coming off. Also this ring could be uncomfortable and cut off blood flow to the penis if too tight Also all men do not have the same size penis, so even though this ring was comfortable for one user, a user with a larger penis would find it uncomfortable. It is also more complicated in use than the Secure Condom.

Foggia Patent; This patent of a condom that has some rubber parts that encircle the scrotum as well as an inner seal of smaller diameter than the rest of the condom. Otherwise the configuration is the same as regular condoms. This condom is difficult to manufacture due to the contrivances attached to a regular condom. This condom could be difficult to use in respect of pushing the penis thru the small seal, and very uncomfortable having a strap around the scrotum, even one of the same material as the rest of the condom. The Secure Condom does everything that this invention does and more without some awkward contrivances to contend with. It will also be much cheaper and easier to produce.

Wheeler Patent; This patent has a flap of similar material to the condom glued or otherwise attached at the open end of the condom with a smaller opening than the remainder of the condom which otherwise resembles a regular condom of prior art. In this invention one embodiment has the rest of the condom rolled up on an applicator ring, however the smaller circumferance compressive retention means is not stretched. The drawing of this, FIGS. 21 and 22 on his patent shows this smaller area not stretched onto the ring. He doesn't say how he got the rest of the condom on the ring without getting this part on, but that must be a mystery. Anyway it would be difficult to install this condom by forcing this small opening over the penis of the user. With the Secure condom the smaller diameter area of the condom is completely rolled upon the installation ring so that at the closed end of the condom, making it very easy and effort-less to install. With the entire area of the shaft of the penis being tightly fit with the Secure condom, not only will this prevent leakage, but will keep the condom on more securely. These condoms will be packaged in soluable lubricant which will make the production of the Secure Condom much simpler to make but also simpler to use. One embodiment of Wheelers invention is having a strip at the open end that is covered with ad-hesive so that the condom can be overlapped to make it tight at the open end. Again this condom would be difficult if not impossible to have lubricant packaged along with the condom or the lubricant would get on the adhesive strip off the hands when trying to attatch the adhesive to itself thereby causing the adhesive to not stick cancelling this benefit. If the condom is not lubricated, you have lost the benefit of the sensitivity of having lubrication in the condoms. The Secure Condom has none of these limitations. It is much simpler to produce than the above inventions. It can be lubricated inside and outside when it is packaged. It can be installed very simply even in the dark since the installation ring will have a larger circumferance on the front side which will make it impossible to roll it off the wrong way. It has an enlarged area which will give the head of the penis the sensitivity of not even having a condom installed and it will not come off by accident if the correct size Secure Condom has been chosen for the particular size penis that it is used on. These condoms will come in different sizes to fit different size penis. The main advantages of Secure Condoms is their simplicity to produce, and to use. Another main advantage is the fact that lubricated inside and outside when packaged. Another big ad-vantage is the enlarged area at the closed end of the Secure Condom that lets the sensitive head of the penis slide freely within the condom during intercourse without fear of it coming off, The sensitivity enjoyed by the user of Secure Condoms is as near as it is possible to achieve when using Condoms.

There is a desperate need for condoms to prevent disease and unwanted pregnancies, however the prior art leaves much to be desired. When the condoms are an improvement over old type condoms, yet the methods that they have come up with are not economical to produce, not practical in use or too difficult to use. The Secure Condoms design is economical to produce because they will be manufactured using current procedures. The Secure Condom will be welcomed because it is lubricated inside and out to give as much sensitivity as is possible in a condom. The enlarged area will give a feeling of freedom that current condoms do not have. In fact Secure Condoms will feel to the user as though he does not have a condom on at all. The Secure Condoms will have the smaller diameter section of the Condom rolled upon the installation ring which is grooved to hold the condom. This installation ring will have one side that is of larger diameter than the other side of the groove to indicate that this side is the front of the condom, thus it will be easy to install even in the dark. The enlarged area of The Secure Condom will not be rolled up on the installation ring but will extend thru the ring. There will be no interferance of the part of the condom rolled up on the ring so that a penis can freely enter the enlarged area thru the installation ring.

To use these Secure Condoms. 1. Remove from package. 2. find which side of the ring is larger; this is the front. 3. push your penis thru the ring into the enlarged area. 4. Roll the part of the condom that is on the ring, off the ring and all the way back to the base of the penis. 5. slide the instal-lation ring off over the nose of the penis and discard. The Secure Condom is now installed.

Wilks patent is the same as contemporary state of the art condoms. The only difference is that the Wilks Patent is double thick and has holes at the open end to equalize the pressure of the outer and inner condoms. There is nothing about this Condom to aid in preventing the condom from coming off during intercourse if it is lubricated on the inside for better more natural sensation.

The Secure Condom has several advantages over the Wilks Patent. The Secure Condom has an enlarged area for the first 2 inches at the closed end which allows movement inside the condom of the Glans Penis, which is the sensitive area of the Penis. The Secure Cond-dom will be packaged in water soluable lubricant so it will be lubricated inside and out for a more natural sensation. Since it will have a smaller area behind the enlarged area that will not go easily over the head of the penis, there is no fear that the secure condom will come off during intercourse. The Secure condom will have a ring upon which the smaller circumferance area of the cond-om will be rolled. This ring will be made of plastic or other strong material and will have an inside diameter large enough for the penis will be able to enter the enlarged area of the condom un-impeded. After the penis is pushed thru the ring, the part of the condom that is rolled up on the ring will be rolled off the ring and unrolled all of the way to the base of the penis. At this time the ring will be slid off over the head of the penis and discarded. The Secure Condom is now installed.

Secure Condoms will come in different sizes, since all men are not endowed equally. Cardboard gauges will be available at no charge, so a man can determine what size condom is right for him. The ring for each size condom will be the same inside diameter as one of the gauges. The condoms will also come in different sizes to match the gauges.

### SECURE CONDOMS

### SUMMARY

The Secure Condom addresses all of the weaknesses of condoms of prior art. A list of these weaknesses follows.

1. condoms accidentally coming off during sexual intercourse.

2. condoms splitting from being too tight.

3. condoms coming off if lubricant is used.

4. lack of sensitivity due to being too tight around the sensitive area known as the Glans Penis.

Secure condoms come in various sizes to fit male members of various sizes. The Secure Condom fits the male members quite tight from the base of the penis to the rear of the head of the penis so that some effort is required to remove this condom over the head of the penis, therefore it will not come off due to the activity of sexual intercourse. (1),(3)

Secure Condoms have an enlarged area for about two inches at the closed end of the condom which is somewhat larger than the head of the penis. This enlarged area gives a sensation of not having a condom installed, and in conjunction with the fact that these condoms are lubricated inside and out, the sensation is the same as no condom at all. (2), (4)

Secure condoms will be packaged rolled up onto the installation ring with water soluable lubricant. Since there is no fear of these condoms coming off, being lubricated inside and out is no problem, and with the enlarged area at the closed end there is no fear of splitting. (1), (2), (3), (4)

Secure condoms will come in various sizes since male members come in various sizes. Cardboard gauges with holes similar to the holes in the different sized installation rings will make it easy to determine the correct size condom for each user. these gauges will be free and available wherever secure condoms are sold. The installation rings will have the condoms small circumferance area rolled up on the groove on the outside of the ring. The enlarged part of the condom will be pushed thru the ring before packaging. The installation ring will have the front side of the groove of larger circumferance to prevent the condom from being rolled off in the wrong direction.

The manufacture of Secure Condoms will use the same methods of manufacture as the condoms of prior art most in use at present. Other than the cost of the installation ring, there is no reason that these condoms will be more expensive than the condoms of prior art.

What I claim is:

1. An improved condom with none of the drawbacks of condoms of prior art, namely said condoms of prior art coming off during sexual intercourse, or said condoms of prior art splitting from being too tight, Secure Condoms will have an area extending from the open end of said Secure Condom to within approximately two inches of the closed end of said Secure Condom of a smaller circumferance than the shaft of the male member said Secure Condom will fit, thereby preventing said Secure Condom from coming off accidentally, and an enlarged area of larger circumferance of said Secure Condom extending from the closed end of said Secure Condom for a distance of about two inches, said enlarged area of larger circumferance than the head of the male member said Secure Condom will fit to prevent said Secure Condom from splitting due to being too tight, said Secure Condom will have an installation ring to facilitate installing said Secure Condoms on male members, said Secure Condoms will come in various sizes to closely fit male members of various sizes comprising
A. a thin rubber, plastic, or other suitable material condom, open on one end and closed on the other end, with area (B) of said Secure Condom extending from said open end to within approximately two inches from the closed end of said Secure Condom, said area (B) being of smaller circumferance than the circumferance of the shaft of the male member said Secure Condom will fit, and area (A) that will extend from the closed end of said Secure Condom for a distance of approximately two inches of a larger diameter than the circumferance of the head of the male member said Secure Condom will fit to prevent said Secure condom from splitting due to tightness, said Secure Condoms will have an installation ring to facilitate installing said Secure Condoms on male members, said Secure Condoms will be available in various sizes to fit male members of various sizes
B. said installation ring will be formed of hard plastic or other suitable material, said ring having a hole thru the center of large enough circumferance for the head of said male member to easily enter, said installation ring will have a groove around the outside circumferance that will allow the area (B) of said secure condom to be rolled up and stretched onto said installation ring, said groove will have one side of larger circumferance than the other side of said groove on said installation ring to prevent rolling said area (B) off said installation ring in the wrong direction, said area (A) of said secure condom will not be rolled up onto said Installation ring, but will extend thru said installation ring, said installation rings will come in various sizes matching the size of said secure condoms, said secure condoms will come rolled up on the correct size said installation rings, packaged in water soluable lubricant, whereas said condoms of prior art are uniform in shade from, the closed end to the open end of said condoms of prior art, and said condoms of prior art are of one size, said condoms of prior art do not fit equally all male members, since all male members are not of one size, said secure condoms come in various sizes, large sizes for male members that are large, small sizes for male members that are small, and a number of sizes in between, said area (B) of said secure condoms will fit tightly enough that it will take some small effort to remove said secure condoms over the head of the male member, such effort will not be provided by sexual intercourse, said enlarged area (A) of said secure condoms will give the sensation of having sexual intercourse without using a condom, said secure condoms can be packaged in water soluable lubricant without the fear that said secure condoms will come off accidentally, said lubricant will add to the natural feeling of having sex without a condom installed, said condoms of prior art could nearly guarantee coming off when lubricated unless a male member was quite large, and then the possibility of said condom splitting was the worry, not coming off by accident. to facilitate choosing the correct size said secure condom, cardboard gauges will be available wherever said secure condoms are sold, said gauges will have holes similar in size to the holes in said installation rings so potential user of said secure condoms can determine the correct size for themselves, said gauges will be free, said secure condoms will use the same methods of manufacture that is most prevalent in condoms most in use presently, said installation ring should add only a few pennies to the cost, therefore said secure condoms cost of production should be similar to present condoms of prior art. said secure condoms simplicity of manufacture and use will make them far superior to condoms of prior art
